(19) United States
(12) Patent Application Publication
(10) Pub. No.: US 2010/0004583 A1
(43) Pub. Date: Jan. 7, 2010

(54) Hydrophobic Circuit Board Coating of Electrotransport Drug Delivery Devices

(75) Inventors: Rodney M. Panos, Redwood City, CA (US); Steven Rabin, Mountain View, CA (US); Robert K. Lowry, Indialantic, FL (US); Aimee Raymond, Milpitas, CA (US); Arthur Jonath, Portola Valley, CA (US)

Correspondence Address: Diehl Servilla LLC, 77 Brant Avenue, Suite 210, Clark, NJ, 07066, US

(73) Assignee: ALZA Corporation, Fremont, CA (US)

(21) Appl. No.: 12/494,406

(22) Filed: Jun. 30, 2009

Related U.S. Application Data
(60) Provisional application No. 61/077,249, filed on Jul. 1, 2008.

(51) Int. Cl. A61N 1/30; A61B 19/02; B05D 5/00

(52) U.S. Cl. 604/20; 206/438; 427/2.1

(57) ABSTRACT

The present invention encompasses an improved electrotransport drug delivery device utilizing a hydrophobic modifier to prevent moisture condensation. Use of a hydrophobic modifier increases the hydrophobicity of coated parts of the device, reducing the amount of moisture uptake of the device during storage in a high humidity environment. In a further embodiment, a package of the improved electrotransport drug delivery device is provided. In addition, a method of reducing moisture condensation to an electrical device and a method of preserving an electrical device for an electrotransport drug delivery device are provided.

### CROSS REFERENCE TO RELATED APPLICATIONS

The present application claims the benefit of U.S. Provisional Application Ser. No. 61/077,249, filed Jul. 1, 2008, the entire disclosure of which is incorporated herein by reference.

### TECHNICAL FIELD

The subject matter described herein relates to electrotransport drug devices, and more specifically, to an integrated iontophoretic drug delivery system stored in a high relative humidity environment before therapeutic use. The high humidity during storage of the device poses particular challenges to prevent malfunctioning of its electronic circuitry.

### BACKGROUND OF THE INVENTION

Electrotransport devices are commonly used for transdermal delivery of drugs. The term “electrotransport” as used herein refers generally to the delivery of an agent, i.e. drug through a membrane, such as skin, mucous membrane, sclera or nails. A detailed definition of electrotransport can be found in U.S. Pat. No. 6,881,208; incorporated herein by reference in its entirety.

In short, electrotransport devices comprise at least two electrodes that are in electrical contact with some portion of surface of the body, through which the agent is to be delivered. The “donor” electrode is the electrode from which the agent is delivered into the body. Hence, electrotransport delivery systems require at least one reservoir or source of the agent to be delivered to the body. Such reservoirs are electronically connected with and positioned between the electrodes and the body surface with the agent reservoir in close physical contact with the donor electrode. The agent reservoirs are composed of matrices such as hydrogels. Hydrogels are often utilized as matrix material, because water is the preferred liquid solvent of the agents and has excellent biocompatibility with skin and mucosal membranes. Hydrogels quickly absorb water and subsequently possess high equilibrium water content. Finally, in order to drive a flux of agent into the body, an electronic current is needed to flow between the electrodes and is regulated by an electronic controller.

In small self-contained electrotransport delivery devices to be worn on the skin all the above components are encapsulated in a container including the hydrogel and a printed circuit board (PCBs) as part of the electronic controller system. The IONSYS™ product is such a small self-contained electrotransport drug delivery device for analgesic drug agents. The IONSYS™ device is shipped in a sealed pouch to the site of patient administration of the agent in order to assure that the hydrogel is sufficiently hydrated before usage of the device. Since the device is sealed into a pouch, it is exposed over the duration of storage to relative high humidity values of close to 100%. The exposure of the device to such high humidity levels possesses the potential for malfunctioning, i.e., self-initiation or non-initiation of the delivery process due to corrosion or deterioration of the electronic circuitry.

Conformal coatings are commonly used to protect PCBs or parts thereof from moisture. The coatings are typically made of materials such as epoxies, silicones, or urethanes. In general, when working to prevent corrosion, these materials are applied to specific areas on the PCBs to prevent access of moisture or corrosive gases to the metallic surfaces or electronic components of the PCBs. For a general discussion on conformal coatings the “Handbook of Polymer Coatings for Electronics—Chemistry, Technology and Applications, 2^{nd} Edition” by J. J. Licari et al. (1990) should be consulted. The percentage of water absorption depends on the particular conformal coating with most coatings absorbing water to varying degree. Yet, the effectiveness of a coating is not only determined by its percent absorption of water at constant temperature over a period of time, but also by the rate of water-vapor migration through the coating. More often, the coatings are not chemically inert, but rather directly influence the corrosive interactions of water or other contaminants with components of the PCB circuitry. Therefore, finding an effective PCB coating for protection of a particular electronic device against water moisture and other environmental moist or vaporous contaminants is not a trivial task, and depends on multiple factors, such as temperature, humidity level, coating thickness and composition, length of exposure.

Not surprisingly, the art has produced little in the field of conformal coatings applied in the field of electronic drug delivery devices, since environmental factors experienced by these devices are very different from environmental factors experienced by electronic devices utilized in other applications. In particular, the application of conformal coatings to the electronic circuitry of an integrated electrotransport drug delivery system such as IONSYS™ has not been described.

### SUMMARY OF THE INVENTION

The following aspects and embodiments thereof described are intended to be exemplary and illustrative, not limiting in scope.

The present application relates to electrotransport drug delivery devices for delivering a beneficial agent through the body surface of a patient. More specifically, the present invention provides an improved electrotransport device that includes an electrical circuit coated with a hydrophobic coating. Even more specifically, the hydrophobic coating reduces water droplet condensation on the circuit, but does not interfere with the solderability of electrical components or conductive traces of the circuit. This prevents malfunctioning and corrosion of the electronics of the device due to moisture condensation, while the coating and its application to the circuit does not negatively interfere with the overall manufacturing process or the operability of the device. The present invention provides such electrotransport devices and methods of making and using such electrotransport devices.

In one aspect, an electrotransport drug delivery device is provided. The device has a first electrode assembly, a second electrode assembly, and an electrical circuit that is electrically connected to the first electrode assembly and the second electrode assembly. At least one of the electrode assemblies contains a beneficial agent to be delivered through the patient's body surface. The electrical circuit drives the electrotransport of the beneficial agent and has a hydrophobic coating and an electrical component under the hydrophobic coating. The hydrophobic coating reduces water droplet condensation on the electrical circuit with the electrical component being still solderable.

The thickness of the hydrophobic coating is preferably less than 10 microns and more preferably being between 2 to 5 microns to facilitate the ease of applying the coating to the device, since it dries quickly and forms a thin uniform transparent layer after coating the device. In addition, the thin coating layer allows electrical circuit elements to be soldered to the electrical circuit such that the electrical component forms good electrical contact with the electrical circuit elements. The present invention therefore permits soldering of the electrical components even after coating parts of the electrotransport device compared to standard conformal coating that can only be applied after fully assembling and soldering the electric circuit.

Furthermore, the coating of the present invention reduces the amount of water condensing on the coated electric circuit of the electrotransport device by increasing the hydrophobicity of, for instance, the PCB of the electrotransport device. Reducing the variability of hydrophobicity among PCBs from various manufacturers made from different materials and utilizing different solder mask chemistry encompasses another benefit of the coating. The contact angles of a variety of different PCBs as a measure of hydrophobicity are identical within a 1% range after being coated as described in the present invention.

In one embodiment of the device, the coating includes fluorinated polymers. An added benefit of using fluorinated polymers includes that these polymers are chemically inert and exhibit no out-gassing of organic volatiles as standard conformal coating such as epoxies, silicones, and urethanes are known to do. One finding of the present invention entails that an increased amount of volatiles in the headspace of a package of the electrotransport device leads to larger moisture condensation on vapor-exposed surfaces inside the package used of storing the device. Water droplets forming on components of the electric circuit in particular result in elevated corrosion and malfunctioning of the device.

In another embodiment the hydrophobic coating also shields the electronic circuitry of the device from contact with corrosive organic vapors that are, for instance, emitted by the power source of the device.

Another advantage of the present invention is that the hydrophobic coating prevents short-circuiting or bridging of oppositely charged contacts due to higher surface tension and a larger water contact angle of the coated PCB. We discovered that hermetic sealing of the PCB was not necessary to yield this advantage. Instead applying a single layer of hydrophobic coating of fluorinated polymers resulted in a sufficiently large water contact angle. Typically, the thickness of the fluorinated polymer coating was less than 10 microns. We also discovered that additional layers of coating had no effect on either the contact angle or the amount of moisture uptake of the PCB. As a result, a single layer of hydrophobic coating is sufficient for the present invention.

In various embodiments of the device the coating includes fluorinated polymers as hydrophobic coats with a thickness of less than 10 microns.

Furthermore, methods are presented that preserves the functionality of an electrotransport drug delivery device by preventing dehydration of its water-containing reservoirs as well as corrosion of its electronic parts due to for instance a high humidity environment during storage in a water-vapor-tight enclosure. Utilizing hydrophobic modifiers to prevent water condensation in 100% relative humidity environments during long-term storage almost completely eliminates the malfunctioning risk of the device. In particular, this prevents corrosion of various electrical components and conductive traces on the PCBs of the electrotransport device by preventing liquid water to accumulate, yet allowing soldering of the components.

In another aspect, a method of reducing moisture condensation to an electrical arrangement of an electrotransport drug delivery device is provided. The method includes coating a hydrophobic modifier on a PCB of the device. The hydrophobic modifier thereby forms a hydrophobic coating such that an electrical component under the hydrophobic coating is still solderable. The hydrophobic coating also reduces condensation of water droplets, for example, on the PCB. An electrical controller is made with the PCB that is used in the electrotransport device to drive the electrotransport flow of a drug from a drug reservoir in the device through the body surface of a patient.

A method of preserving an electrical arrangement of an electrotransport drug delivery device is also presented. The method includes coating a hydrophobic modifier on a PCB of the device. The hydrophobic modifier forms a hydrophobic coating, which reduces condensation of water droplets on the PCB. An electrical controller is made with the PCB that is used to drive via electrotransport the drug flow form a drug reservoir in the device through the body surface of a patient. An electrotransport device is made having the coated PCB and at least one hydrogel reservoir. The electrotransport device is then enclosed in a water-vapor-tight enclosure such that water vapor can transfer water vapor in the enclosure can transfer between the hydrogel reservoir and the head space of the enclosure. This allows the water vapor to equilibrate to saturation at room temperature.

In yet another aspect of the present invention, a packaged electrotransport drug delivery device is provided. The electrotransport device has a first electrode assembly, a second electrode assembly, and an electrical circuit electrically connected to the first electrode assembly and the second electrode assembly. At least one of the electrode assemblies contains a beneficial agent in a hydrogel reservoir to be delivered through the patient's body surface. The electrical circuit drives the electrotransport of the beneficial agent and has a hydrophobic coating and an electrical component under the hydrophobic coating. The electrical component under the hydrophobic coating that reduces water droplet condensation on the electrical circuit is still solderable. Furthermore, the electrotransport device is enclosed in a water-vapor-tight enclosure such that water vapor in the enclosure can transfer between the hydrogel reservoir and the headspace of the enclosure. This allows the water vapor to equilibrate to saturation at room temperature.

In this embodiment, the device and its electric components are exposed to relatively high humidity levels due to storage of the device in a water-vapor-tight enclosure that prevents dehydration of the water-containing reservoir of the electrotransport device.

Thus, with the present invention, one will be able to store an electrotransport drug delivery device in a high humidity environment, yet eliminate problems and malfunctioning of the device due to corrosion of its electrical parts in such an environment. Thus, the overall safety of the device is increased. Furthermore, as disclosed herein, applying the hydrophobic coating can be readily integrated in an already existing manufacturing process of the device because of the ease of handling the hydrophobic coating and its low material cost, while not negatively impacting the assembly of the device that includes for instance the soldering of electric circuit elements to the coated PCB.

### BRIEF DESCRIPTION OF THE DRAWINGS

Further features and advantages to the exemplary aspects and embodiments described above will become apparent from the following and more particular description of the preferred embodiments of the invention, as illustrated in the accompanying drawings, and in which like referenced characters generally refer to the same parts or elements throughout the views, and in which:

FIGS. 1A-D are images of water droplets on surfaces with different hydrophobicity. The images clearly display the contact angle θ of each droplet;

FIG. 1E is a schematic bottom-view of a printed circuit board of an electrotransport drug delivery device showing the conductive traces on the board;

FIG. 1F is a schematic cross section of a printed circuit board of a electrotransport drug delivery device displaying the non-conductive substrate, electrical contacts, and solder mask with a water droplet condensed between the contacts;

FIG. 1G is a schematic cross section of a printed circuit board of a electrotransport drug delivery device displaying the non-conductive substrate, electrical contacts, solder mask, and hydrophobic coating with a water droplet condensed between the contacts;

FIG. 2 is a graph illustrating the moisture mass uptake in the presence and absence of DME as well as the water contact angle of PCBs treated with different hydrophobic coatings;

FIG. 3 is a graph illustrating the moisture mass uptake of surfaces with varying hydrophobicity in the presence and absence of dimethoxyethane (DME) in relation to their water contact angle.

FIG. 4A is a graph illustrating the moisture mass uptake of PCBs from certain manufacturers having different solder masks with and without hydrophobic coating;

FIG. 4B is a graph illustrating the contact angle of water on the surface of PCBs from certain manufactures having different solder masks with and without hydrophobic coating;

FIG. 4C is a graph illustrating the moisture mass uptake of PCBs from certain manufacturers having different solder masks in the presence of DME with and without hydrophobic coating;

FIG. 5A is a graph illustrating the total uptake of moisture mass in the presence and absence of DME for PCBs having different solder masks and at various temperatures;

FIG. 5B is a graph illustrating the total uptake of moisture mass in the presence of varying DME amount for PBC having different solder masks and at various temperatures;

### DETAILED DESCRIPTION OF THE INVENTION

The following detailed description should be read with reference to the drawings, in which like elements in different drawings are identically numbered. The drawings, which are not necessarily to scale, depict selected exemplary embodiments for the purpose of explanation only and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention. It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

For purposes of this application, reference to the term “package” or “packaging” will be understood to also include reference to “storage” or “storing” and vice versa.

Further, all publications, patents and patent applications cited herein are hereby incorporated by reference in their entirety.

Finally, as used in this specification and the appended claims, the singular forms “a, “an” and “the” include plural referents unless the content clearly dictates otherwise. Thus, for example, reference to “an active agent” includes two or more such agents; reference to “a hydrophobic coating” includes two or more such coatings and the like.

### DEFINITIONS

Fluorocarbons are carbon-fluorine compounds that often contain other elements such as hydrogen, chlorine, or bromine. Common fluorocarbons include chlorofluorocarbons (CFC) and related compounds (also known as ozone depleting substances including hydrochlorofluorocarbon which is a CFC substitute). Fluorocarbons are polymers and are organic compounds containing fluorine directly bonded to carbon. The ability of the carbon atom to form a large variety of structural chains gives rise to many fluorocarbons and fluorocarbon derivatives. For example, a famous fluorocarbon is DuPont's Teflon. Fluorocarbons are also used in fishing line and myriad precision plastics applications. The material is tough, non-contaminating and is an electrical insulator. Fluorocarbon compounds are also used in highly precise lubrication applications.

The contact angle is the angle at which a liquid/vapor interface meets the solid surface. The contact angle is specific for any given system and is determined by the interactions across the three interfaces. Most often the concept is illustrated with a small liquid droplet resting on a flat horizontal solid surface. Ideally, the droplet should be as small as possible because the force of gravity, for example, can actually change the above-mentioned angle. The shape of the droplet is determined by the Laplace equation. The contact angle plays the role of a boundary condition.

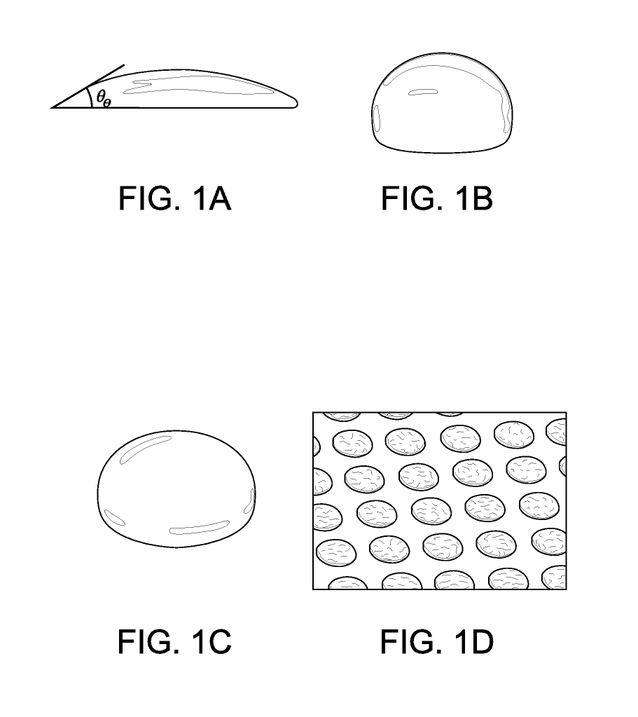

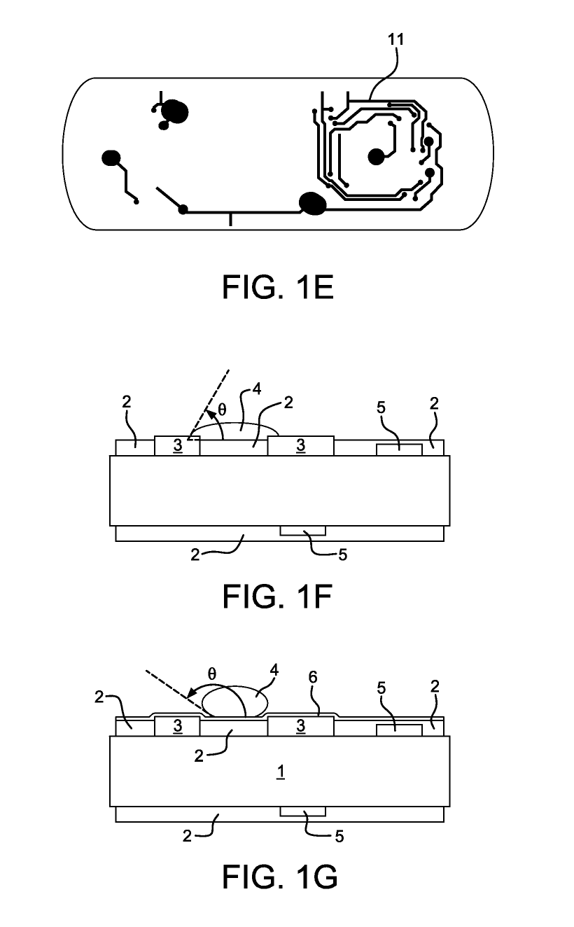

On extremely hydrophilic surfaces, a water droplet will completely spread (an effective contact angle of 0 degree). This occurs for surfaces that have a large affinity for water (including materials that absorb water). On many hydrophilic surfaces, water droplets will exhibit contact angles of 10 degree to 30 degree. On highly hydrophobic surfaces, which are incompatible with water, one observes a large contact angle (70 degree to 90 degree). Some surfaces have water contact angles as high as 150 degree or even 180 degree. On these surfaces, water droplets simply rest on the surface, without actually wetting to any significant extent. These surfaces are termed superhydrophobic. Images of water droplets on such surfaces with varying hydrophobicities that display a range of contact angles θ are shown in FIGS. 1A-D. The contact angle thus directly provides information about the interaction energy between the surface and the liquid.

Current approaches for protecting the electronic circuitry of an electrotransport drug delivery device from high relative humidity in an enclosed environment such as a storage pouch are problematic. The presence of water as part of the device is essential for its operability, yet can lead to deterioration and corrosion of its electronic components, and ultimately to device failure due to electronic malfunctioning.

The present invention provides an improved electrotransport drug delivery device, a method for reducing moisture condensation to an electrical arrangement of an electrotransport drug delivery device, and a method of preserving thereof. The improvement provides coating the electrical circuit of the electrotransport drug delivery device with a hydrophobic coating to reduce water droplet condensation on components of the circuitry. The resultant electrical arrangement of an electrotransport drug delivery device improves on protection against corrosion of its electrical components, thereby increasing the shelf life of such devices, while providing ease of manufacturing without altering the electrical characteristics of the electrical device. This is achieved by coating the electrical arrangement (including for instance a PCB) with a thin hydrophobic coating that allows for soldering electrical arrangement after the application of the coating. Preferably, the coating is either applied to the PCB before reflow or the PCB assembly after reflow. This is beneficial as it allows for easily integrating the coating process in the overall manufacturing process of the electrotransport device without incurring significant costs.

Structurally, the electrotransport drug delivery device includes at least two separate electrode assemblies, labeled first and second, respectively. The device is self-contained and can have essentially any convenient size or shape, whether square, oval, circular, or tailored for a specific location on the body. A self-contained device implies that all components necessary for delivering an agent via electrotransport are assembled inside a small-sized, easy-to-handle container. The device furthermore can be flexible and easily conform to a body surface. At least one of the electrode assemblies contains an agent that is to be delivered through a body surface of the patient.

The electrode assemblies are electronically connected via an electrical circuit schematically shown in FIGS. 1E and 1F. The electric circuit pathway is relatively thin and preferably has conductive traces 11 printed, painted or otherwise deposited on one or both sides of a thin substrate or board 1. The maximal thickness of the nominal thick printed circuit board is preferably between 0.5 mm and 0.82 mm, more preferably between 0.61 mm and 0.082 mm. With respect to PCB trace spacing, on IONSYS™, spacing between traces is typically 0.5 to 1 mm, with a few traces as close as 0.25 mm. On a PCB of another embodiment of an electrotransport device, spacing between traces will be typically 0.22 mm to 0.5 mm. In particular, the spacing between pads on an employed integrated circuit will be 0.22 mm. Furthermore, one or both sides of the PCB is covered by a solder mask 2 that is a coating applied over selected areas of the board, thereby permitting soldering only of the exposed, uncoated areas 3. Usually only pads that are for mounting or the attachment of electric circuit elements are exposed solder contacts on the PCB. Therefore, parts of the conductive traces 5 are covered by the solder mask 2.

In addition to a power source, the circuit may also include one or more of the following electric circuit elements which control the level, waveform shape, polarity, timing and other physical properties of the electronic current applied by the device, including for example, control circuitry such as a current controller, an on/off switch, and/or a microprocessor adapted to control the current output of the power source over time.

The electrode assemblies can be physically separated form one another by an electrical isolator, and form therewith a single self-contained unit. Typically, the electrode assembly is composed of an electrode and an adjacent reservoir that either contains the therapeutic agent and/or an electrolyte. Electrodes may contain metal foils, or a polymer matrix loaded with metal powder, powdered graphite, carbon fibers, or any other suitable electrically conductive material. The reservoirs can be polymeric matrices or gel matrices adapted to hold a liquid solvent. Aqueous-based or polar solvents, especially water, are generally preferred for delivering agents across a body surface. When using an aqueous-based solvent, the matrix of reservoirs preferably has a water retaining material and contains more preferably a hydrophilic polymer, such as a hydrogel. Natural or synthetic polymer matrices may be employed.

In a typical embodiment of the electrotransport device, the agent reservoir or reservoirs contain neutral, ionized, or ionizable supply of the agent or multiple agents to be delivered, while it can also contain a suitable electrolyte such as, for example, sodium chloride, potassium chloride or mixtures thereof. The reservoirs of the electrotransport drug delivery device must be placed in agent transmitting relation to the body surface of the patient, through which the agent is delivered. Usually this means the device is placed in intimate contact with the patient's skin as one possible body surface. Practice of this application is not to be limited to any particular therapeutic agent. Generally, the combined body surface contacting area of the electrode assemblies can range from about 1 cm² to about 200 cm², but typically will range from about 5 cm² to about 50 cm².

After manufacturing, a self-contained electrotransport drug delivery device is typically packaged within a water-vapor-tight enclosure. The enclosure acting as a container could be made from a foil pouch large enough in size to hold the device. The pouch material is selected from materials known in the art. It is preferred that the pouch material is self-sealable, light in weight, and acts as a barrier to the water vapor contained within the pouch. For example, suitable pouch materials are disclosed in U.S. Pat. Nos. 5,077,104 and 5,268,209, which are hereby incorporated in their entirety by reference.

Packaging a small self-contained electrotransport drug delivery in a tightly sealed foil pouch that is impervious to water vapor assures maintaining a high moisture level in the hydrogels. Sufficient degree of moisture in the hydrogel is necessary to ensure normal functionality of the electrotransport device. Some water does escape the gels inside the pouches as vapor, which over a short period of time leads to almost 100% relative humidity in the pouches. Temperature and other changes in the pouch environment cause the water vapor in the pouch atmosphere, also referred to as headspace, to condense. The surface of the electronic circuit and components thereof, such as a printed circuit board (PCB), comprise one potential place that the water can condense on.

How much water condenses on a surface patch of, for instance, the PCB depends on how hydrophilic that particular surface area is in comparison to other water vapor exposed surfaces inside the pouch. Generally, the sorpted amount of water that is absorbed as bulk water or adsorbed on a particular surface area is called the moisture mass uptake and is measured in microgram of water per millimeter² of the surface area. The total moisture mass uptake of, for instance, a PCB is then defined as the sum of the moisture sorption amount over the entire PCB surface.

Another direct measure of hydrophilicity constitutes the contact angle of the various surfaces with the contact angle being a function of the overall surface energy. The contact angle θ of a water droplet 4 on a PCB is shown in FIG. 1F. In the case of PCBs, the surface energy is foremost a function of the solder mask 2 used to cover the conductive traces 11 on the PCBs as well as the composition and manufacturing process of the PCBs.

Nowadays, the PCB industry is moving toward water-based solder masks and away from organic solvent-based masks for environmental reasons. Since water-based solder masks are naturally more hydrophilic than organic solvent-based masks, PCBs subsequently accumulate higher amounts of condensing water vapor, which in turn makes them more prone to corrosive processes.

For a small self-contained electrotransport drug delivery device, such as IONSYS™, the utilization of water-based solder masks on the PCBs has caused more water to collect in areas on the board where it is undesirable due to the potential of corrosion. Since certain electrical components on the board are under electrical bias, the presence of liquid condensed water can accelerate corrosion leading to self-initiating failure or other malfunctioning of the device.

Typically, solder masks are photosensitive protective dielectric laminates that are placed on the outer surfaces of the printed circuit board prior to the assembly process of electric circuit elements onto the board. Examples of solder masks include “Taiyo PSR4000BN(HV)” by Taiyo America Inc. NV, “Ciba Probimer 52” by Ciba Geigy, “Coates ImageCure XV501T-4” by Coates Circuit Products (Bath, UK), or one of many other chemicals used in the industry with the desired dielectric and photosensitive characteristics. After laminating the board the solder mask is then polymerized in areas that are exposed to a light source through a mask. Upon removal of the unexposed solder mask, the remaining polymerized solder mask is dried and cured, usually thermally.

In addition, we have found that one change in the environment that increases water condensation is the leakage of organic or acidic vapors from components of the device resulting in water vapor from the enclosed headspace to condense on various surfaces inside the pouch container. Removing leaked vapors will eliminate this effect resulting in less moisture condensation and in turn would reduce or prevent corrosion of the device. For example, 1,2-dimethoxyethane (DME), a solvent used as an electrolyte in the lithium battery of the IONSYS™ device, was shown to slowly leak from the battery and cause increased water condensation, and in part on the PCB of the device. In general, any organic volatile compound present in parts of the device can potentially contribute to the overall vapor pressure, effectively forcing water to condensate. Our experiments have demonstrated that in the presence of DME vapor, ˜30-50% more water vapor can condense out relative to systems without DME.

Additionally, reducing the exposure time of the electronic components to acidic vapors will eliminate another possible cause of their corrosion. Despite the operating temperatures of the system (10°-40° Celsius) being relatively “benign”, the electronic components of the electrotransport device are actually situated in a harsh environment. The acidic vapor enhances the chemical reactivity of moisture condensing on various surfaces of the electrotransport drug delivery device, increasing the rate of corrosion of metallic components of the device and subsequent device failures. Additionally, dissolving ions in the condensate can lead to electric current flow on the PCB due to a voltage applied by a power source, e.g., the battery, during storage of the device, causing malfunctioning of the device.

The list of organic and acidic volatiles that may be present in an electrical system in an electrotransport device includes, but is not limited to, acetone, methylacrylate, methyl or isopropyl cellosolve, acetic acid, 1,2-dimethoxyethane, dimethylformamide, butanol, methoxy isopropyl alcohol, heptane, methyl methacrylate, propylene glycol, toluene, methacrylic acid, 2-hexanone, xylene, 3-methyl hexanone, styrene, trimethyl benzene, propylene carbonate, alpha-methylstyrene, 2-ethoxyethoxy-ethanol, decane, undecane, 2-methylbutanoic acid, (2,4)-di-tert-butylphenol, butylated hydroxytoluene, difluoroethane, propene, methanol, acetaldehyde, isobutylene, methylformate, acetone, t-butanol, 2-methylpropanal, propanol, 2-methylpentane, methacrolein, cyclohexane, butanal, methyl ethyl ketone, ethylacetate, trimethylsilanol, tetrahydrofuran, butanol, 2-pentanone, heptane, (1,4)-dioxane, dichloromethane, dimethylcyclopentane, cyclotetradecene, hexadecene, and carbon tetrachloride.

To mitigate these problems, in an embodiment of the present invention a hydrophobic coating 6 is deposited on the surface of the substrate or board 1 containing the conductive traces 11 of the device circuitry or on top of the solder mask 2 (FIG. 1G). The thickness of coating required is on the order of a few microns. In comparison conformal coating commonly used in protecting electronic circuitry against moisture measure from tens or hundreds of microns (or more) in thickness.

In order to effectively protect the electronics of the electrotransport drug delivery device from corrosion or other moisture-induced malfunctioning, the coating has to yield a contact angle of the treated surface larger than 80 degree and preferably a surface energy of less than 30 dynes/cm. A coating with these properties will significantly reduce water droplet condensation on coated surfaces. Particularly, in a moisture-tight enclosure and high humidity environment of 95% to 98.5%, the coated surface preferably possesses a contact angle larger than 105 degree with a surface energy smaller than 20 dynes/cm. Furthermore, the coating should be transparent and thin in thickness to facilitate soldering of electronic components after applying the coating. Preferably, the coating is only a few microns thick and forms a uniform layer on coated surfaces of the electrotransport device. More preferably, the coating is covering surfaces of the electrotransport device with a layer measuring less than 10 microns thickness throughout the area covered by the coating.

In a preferred embodiment, the hydrophobic coating is formed by depositing a fluorinated polymer composition on the surface of the electrical circuitry of an electrotransport drug delivery device. The coating composition may comprise a liquid non-polar solvent and a dispersant. The solvent may be any suitable non-polar solvent that is liquid at room temperature.

The choice of solvent to dissolve the fluoropolymer typically depends on the specific fluoropolymer. Methods for selecting appropriate solvents are well known in the art. Preferably, the solvent is a hydrocarbon solvent, more preferably a fluorinated hydrocarbon solvent. More preferably, the solvent is a highly fluorinated solvent, especially a branched or unbranched, cyclic or non-cyclic fluoroalkane. Thus, various linear fluorinated solvents can be used as non-flammable solvents. Perfluoroalkanes, semifluoroalkanes, their chlorine- or bromine-introduced derivatives can be also used, though these are not preferable substances when their environmental influence or toxicity is taken into consideration.

Exemplary organic solvents that may be used for dissolving the fluoropolymer include amides (e.g., N,N-dimethylformamide), ketones (e.g., methyl ethyl ketone), alcohols (e.g., methanol), ethers (e.g., tetrahydrofuran), perfluorinated solvents (e.g., a perfluorinated organic solvent available from 3M Company (St. Paul, Minn.) under the trade designation “Fluorinert Electronic Liquid FC-77”), hydrofluoroethers, and combinations thereof.

The solvent for the coating solutions used according to the invention may comprise a fully fluorinated non-branched fluorocarbon having a carbon chain length of 7 or 8 carbon atoms. Such a solvent exhibits a boiling point of about 80° Celsius. According to embodiments of the invention, fluorinated solvents include the Fluorinert® line of fluorinated solvents, FC-71, FC-75, FC-40, FC-70, FC-77 and FC-84, all from the 3M Company. Other fluorinated solvents which may be used include “Vertrel® XF” (C5H2F10) or “Freon TF” from DuPont (Wilmington, Del.), the fluorinated polyethers HT70, HT85, HT90, HT100, HT110, HT135, HT200, HT230, HT250 and HT270, and the perfluorinated polyethers sold as Galden™, all from Ausimont USA, Inc. The Ausimont USA, Inc. solvent designations indicate the boiling point of each solvent. Higher boiling solvents, for example, HT270 and HT250, would form coatings requiring more heat to dry than coatings made with the lower boiling solvents, for example, HT70. The lower boiling Ausimont USA, Inc. solvents more rapidly evaporate when compared to the higher boiling solvents.

Hydrofluoroethers used as coating solvents are preferred due to their reduced environmental impact, and are not expensive, as compared with ordinary solvents, and are rather suitable as a nonflammable solvent for use in the non-flammable hydrophobic coating. Particularly preferred are segregated hydrofluoroethers. Segregated hydrofluoroethers are available as under the trade designation “HFE-7100 Engineered Fluid” and “HFE-7200 Engineered Fluid” from 3M Company. HFE-7100 is a blend of approximately 60% methyl perfluoroisobutyl ether and 40% methyl perfluoro-n-butyl ether, whereas HFE-7200 comprises a blend of approximately 60% ethyl perfluoroisobutyl ether, and 40% ethyl perfluoro-n-butyl ether.

In an alternative, yet less environmentally desirable embodiment, the solvent is perfluorinated. Exemplary solvents include Fluorinert fluorinated solvents available from 3M company, especially FC-75, a perfluorinated C, solvent, CAS No. 86508-42-1, and FC-84, a perfluorinated C, solvent, CAS No. 86508-42-1.

For rapid evaporation during the drying step, the fluorinated solvent should have a boiling point of less than about 120° Celsius at atmospheric pressure. It is believed that the very low surface energy of the fluorinated solvents renders the composition much more effective as a coating composition. The low surface tension of fluorinated solvents effectively wet the substrates much more readily than the conventional aqueous and alcoholic compositions. Useful fluorinated solvents meeting these criteria include hydrofluoroethers (HFEs), hydrofluorocarbons (HFCs), hydrohalofluoroethers (HHFEs) and hydrochlorofluorocarbons (HCFCs). The fluorinated solvents may optionally contain one or more additional heteroatoms, such as nitrogen or oxygen. The fluorinated solvent may be selected from the group consisting of partially-fluorinated alkanes, amines, ethers, aromatic compounds, and mixtures thereof. The fluorinated solvent is non-functional, i.e., lacking functional groups that are polymerizable, reactive toward acids, bases, oxidizing agents, reducing agents or nucleophiles. Preferably, the number of fluorine atoms exceeds the number of hydrogen atoms in the fluorinated solvents. To be non-flammable, the number of fluorine atoms preferably exceeds the number of hydrogen atoms, and more preferably the number of fluorine atoms is equal to or exceeds the sum of the number of combined hydrogen atoms and carbon-carbon bonds. The fluorinated solvents are partially or incompletely fluorinated, i.e. contain at least one aliphatic hydrogen atom. It is preferred that the fluorinated solvent contains at least one aliphatic or aromatic hydrogen atom in the molecule. These compounds generally are thermally and chemically stable, yet are much more environmentally acceptable in that they degrade in the atmosphere and thus have a low global warming potential, in addition to a zero ozone depletion potential, and better solvency properties.

Other fluorocarbon solvents may be used and typically have boiling ranges of from about 30° Celsius to about 250° Celsius, depending upon a number of factors including the length of the carbon chain. At least partially fluorinated solvents are preferred, particularly those fluorocarbon solvents having at least about 20% by weight fluorine atoms per molecule. Solvents exhibiting surface energies of 18 dynes/cm or lower are preferred, with solvents having surface energies of 13 dynes/cm or lower being more preferred and those having 9 dynes/cm being even more preferred. Volatile or non-volatile fluorinated surfactants may be included in the coating for mutations of the present invention.

The density of particles in solvent (solids content) may be any level at which the dispersion is stable and does not significantly coagulate. For use of the dispersion as a coating in an electrotransport delivery device, the solids content may be any level that allows proper functioning over repeated cycles. Preferably, the solids content is less than 10 wt %, more preferably less than 5 wt %, and most preferably less than 2 wt %.

The dispersant may comprise at least one of a fluoropolymer or a fluoropolymer precursor. The fluoropolymer may be dispersed or dissolved in the solvent, or be a liquid at the selected temperature of applying the coating to surfaces of the electrotransport device. Useful polymers may have fluorine on the polymer backbone and/or side chains. Fluoropolymer precursors typically comprise oligomeric and/or monomeric fluorinated organic compounds that have condensable, polymerizable, and/or crosslinkable groups, and may optionally contain one or more curatives (e.g. initiators, hardener, catalysts).

Useful fluoropolymer and fluoropolymer precursor solutions are described, for example, in U.S. Pat. Nos. 4,132,681, 4,446,269, 6,350,306, 5,459,191, and 6,365,276; the disclosures of which are incorporated herein by reference.

Useful solutions of commercially available fluoropolymers and fluoropolymers precursors include, for example, are marketed by 3M company under the trade designations “Novec™ Electronic Coating EGC-1700”, “Novec™ Electronic Coating EGC-1702”, and “Novec™ Electronic Coating EGC-1704”; and fluoropolymer solutions marketed by Cytonix Corp. (Beltsville, Md.) under the trade designations “FluoroPel™ PCF 501A”, “FluoroPel™ PFC 502A”, “FluoroPel™ PFC 504A”, “FluoroPel™ PFC 1340A”, “FluorN™ 561”, “FluorN™ 562”, and “Fluorothane™ ME”. “FluorN™ 561” and “FluorN™ 562” are fluorosurfactants that are 100% solid, non-reactive, high fluorine content and ethylene glycol based polymers.

Useful dispersible fluoropolymers include, for example, those described in U.S. Pat. No. 6,518,352 (Visca et al.); U.S. Pat. No. 6,451,717 (Fitzgerald et al.); U.S. Pat. No. 5,919,878 (Brothers et al.); and PCT patent publication WO 02/20676 A1 (Krupers et al., published Mar. 14, 2002); the disclosures of which are incorporated herein by reference.

Useful dispersions of commercially available fluoropolymers and fluoropolymer precursors include, for example, polyvinylidene difluoride (PVDF) dispersions (e.g., as that marketed by Atofina Chemical (Philadelphia, Pa.) under the trade designation “Kynar 500”); polytetrafluoroethylene (PTFE) dispersions (e.g., as marketed by DuPont under the trade designations “Teflon PTFE Grade 300”, “Teflon PTFE Grade 307A”; or as marketed by Dyneon Corp. under the trade designations “TF 5032 PTFE” or “TF5050 PTFE”); tetrafluoroethylene-hexafluoropropylene-vinylidene fluoride dispersions (e.g., as marketed by Dyneon under the trade designations “THV 220D Fluorothermoplastic” and “THV 340D Fluorothermoplastic”).

Solutions of polymers made from monomers having terminal trifluoromethyl groups are also commercially available. One solution, which can be used to form polymeric hydrophobic coatings according to the invention, is available from the 3M Company as FC-722. Other trifluoromethyl group-containing polymer solutions in fluorosolvents are available from Cytonix Corp. as the PerFluoroCoat™ and FluoroPel™ products lines. The coating solutions used according to embodiments of the present invention comprise fluoropolymers having terminal trifluoromethyl groups. The solutions can be used full strength but may be diluted with a fluorosolvent to form low concentrations of coating polymer. The polymer solution used to make the coatings of the invention preferably have a coating polymer content of from about 0.01% by weight to about 50% by weight.

According to embodiments of the invention, hydrophobic coatings are provided which may preferably comprise, and more preferably consist essentially of, a polymerization product of a substantially non-branched perfluoroalkyl monomer. Coatings according to the invention may comprise polymerized products of monomers having terminal trifluoromethyl groups, including fluorinated or perfluorinated monomers such as hexyl-ethylenically unsaturated monomers, heptyl-ethylenically unsaturated monomers, octyl-ethylenically unsaturated monomers, nonyl-ethylenically unsaturated monomers, decyl-ethylenically unsaturated monomers, undecyl-ethylenically unsaturated monomers, and dodecyl-ethylenically unsaturated monomers. Mixtures of two or more different monomers may also be used and are preferred when it is desired to adjust surface energy properties to precise values. The coatings of the present invention may comprise or consist essentially of a polymerization product of a fluoroalkyl ethylenically unsaturated monomer having a terminal trifluoromethyl group and a carbon chain length of from 3 to 20 atoms, preferably from 6 to 12 carbon atoms in length, and more preferably from 8 to 10 carbon atoms in length. In particular, polymerization products of fluoroalkyl methacrylates are preferred. According to some embodiments of the invention, polymerization products of perfluorohexyl methacrylate, perfluoroheptyl methacrylate, perfluorooctyl methacrylate, perfluorononyl perfluorodecyl methacrylate, perfluoroundecyl methacrylate or perfluorododecyl methacrylate, and mixtures thereof, are preferred. Acrylates of such perfluoroalkyls are also preferred. According to one particularly preferred embodiment, the polymer coating consists essentially of a polymerization product of perfluorooctyl methacrylate. Exemplary materials for making the coatings of the present invention include PerFluoroCoat™ and FluoroPel™, both available from Cytonix Corp., the fluorinated materials FC-722, FX-13, FX-14, FX-189, L-9187, L-9186, “Fluorel™FC2174” and “Fluorel™FC 2181”, all available from the 3M Company, silastic fluorosilicone rubbers from Dow Corning STI identified as LS-2249U, LS-2332U, LS-2840 and LS-2860, and fluorinated materials from DuPont including materials traded under the name Zonyl.

The coating compositions, according to the present invention, can also include functionalized fluoropolymers that have cross-linkable chemical groups, for example, Lumiflon® FE3000, FE4100, FE4200, FE4400, LF100, LF200, LF302, LF400, LF600X, LF710N, LF800, LF910LM, and LF916N, from Asahi Glass Co. (Tokyo, Japan).

The coating compositions of the present invention can include fluorourethanes, for example, those available from Century 2000 Coatings (Alexandria, Va.), “Fluorothane™ ME” available from Cytonix Corp. and those disclosed in U.S. Pat. No. 4,132,681, which is herein incorporated in its entirety by reference. Fluorourethanes comprising polymers of polyisocyanates and fluorine-containing diols are particularly preferred, resulting in good chemical and mechanical properties. These fully or partially fluorinated resins may be used as primers for other coatings of the present invention or as mixtures with polymers and/or monomers according to the present invention.

According to another embodiment, a hydrophobic coating is applied to the electric circuitry board or substrate to form a coating having an exposed surface area populated with at least 30% by area trifluoromethyl groups.

One benefit of utilizing fluoropolymers is the elimination of volatile organic compounds, the low toxicity and acceptable environmental properties (non-ozone-depleting) as well as being nonflammable. In addition, fluoropolymers possess excellent anti-wetting, anti-sticking, anti-migration and anti-corrosion properties. Another benefit includes the transparency as well as thinness of the film allowing for soldering through the film of the underlying PCB after application of the coating. It also contains excellent chemical and solvent resistance as being an inert material. The water absorption and moisture vapor transmission rate (MVTR) are among the lowest of known polymers. The MVTR of Teflon FEP, for example, is 6.3 g/m²/24 hr/mil. Water absorption ranges after 24 hours immersion range for less than 0.01% to 0.03%.

Various fluorinated polymer solutions include 1 to 4 percent fluoropolymer solutions in low boiling point fluorosolvent with a boiling point of 56° Celsius and available from Cytonix Corp. as “FluoroPel™ PFC 501A, 502A, or 504A”. Thin films deposited on surface dry in seconds at room temperature to a surface energy of about 10 dynes/cm. Warming to 90° Celsius for 10 minutes optimizes adhesion and reduces surface energy to about 6 dynes/cm of the cured film. The preferred method of depositing the fluoropolymer solution is dip application. In another embodiment, 2 percent of fluorosilane or fluorophosphates is added to the fluoropolymer solution to enhance adhesion to metal oxide.

In another embodiment, the fluorosolvent has a low boiling point of 135° Celsius and is available from Cytonix Corp. as “FluoroPel™ PFC 1304A”. Thin deposited films dry in a few hours at room temperature to a surface energy of about 10 dynes/cm. Warming to 90° Celsius for 10 minutes optimizes adhesion and reduces the surface energy to about 6 dynes/cm. The preferred method of applying is fluoropolymer solution to the target surface is by spray or spin application. In yet another embodiment, 2 percent of fluorosilane or fluorophosphates is added to the fluoropolymer solution to enhance adhesion to metal oxide surfaces.

In yet another embodiment, the coating contains a 1 to 4 percent fluoroaliphatic polymer in a fluorosolvent having a boiling point of 84° Celsius and is available from Cytonix Corp. under the trade destination “FluoroPel™ PFC 802A”. The thin film dries in minutes at room temperature to a surface energy of 10 dynes/cm. The preferred method of applying is fluoropolymer solution to the target surface is by dip application. In yet another embodiment, 2 percent of fluorosilane or fluorophosphates is added to the fluoropolymer solution to enhance adhesion to metal oxide surfaces.

In yet another embodiment, the coating formulation includes a clear, low viscosity hydrofluoroether solvent that carries a fluorochemical acrylate polymer (e.g., one is available from the 3M Company under the trade destination “Novec™ Electronic Coating EGC-1700”). The solvent is preferably nonflammable, has low toxicity and provides acceptable environmental properties. When applied, the coating dries to a thin transparent uniform film that is insoluble in solvents such as heptane, toluene and water. It possesses excellent anti-wetting and anti-migration properties to all non-fluorinated liquids, including heptane, toluene, acetone, silicone oils and detergents. The film can endure temperatures up to 175° Celsius for prolonged periods without alteration to its physical characteristics, such as water repellency. Boiling point of solvent is 61° Celsius. Solids of the solution range from 0.2% to 2%. Surface energy of film is 11-12 dynes/cm and coating thickness when dip coated is about 1 micron.

In order to tightly control the characteristics of the hydrophobic coating, in yet another embodiment, the fluoropolymer solution may be further diluted with a solvent, e.g., hydrofluoroether solution, i.e. “HFE-7100 Engineered Fluid”.

Alternatively, additives may be incorporated into or polymerized with the coating polymers and monomers used to provide coatings according to the invention having improved toughness, chemical resistance, hardness, softness, processability, elasticity, adhesion, color, texture, thickness and/or UV resistance. Hydrophobic additives are preferred. Chemically resistant additives are preferred. Additives including non-trifluoromethyl-containing reactants and/or monomers may be added in amounts ranging from 1 to about 95% by weight and are described in more detail below.

According to various embodiments, the coating composition can contain two components or the reaction product of the two components, wherein the two components include an alkoxylated tetrahydrofurfuryl acrylate, and a fluorinated monomer. The alkoxylated tetrahydrofurfuryl acrylate can be a propoxylated tetrahydrofurfuryl acrylate, for example, CD611 available from Sartomer Company Inc. (Exton, Pa.).

According to various embodiments, a method of coating of surfaces of the electrotransport device is provided. Possible methods of applying the fluoropolymer solution to the electronic device of the electrotransport drug delivery device include dip, spray, brush, ink jet, pad print, spin or blot application. Other methods include mechanically applying the coating to the PCB via vacuum deposition. This process could be an optional gas phase polymerization prior to the coating fusing to the PCB. The method can involve combining all the components of the coating and then coating the surface with the combined formulation. Alternatively, the method can involve pre-coating a surface to be coated with one or more of the above described components of the hydrophobic coating, and subsequently coating the pre-coated surface with one or more of the remaining components. The method can include hardening or curing one or more components or component mixtures with heat, moisture, or UV radiation, or by drying, the one or more components or mixtures thereof before, or after, coating the remaining one or more components or mixtures thereof onto a surface of the electrotransport device. Optionally, after the coat has dried, the PCB can be heated to 225° Celsius for 3 minutes as an additional curing step.

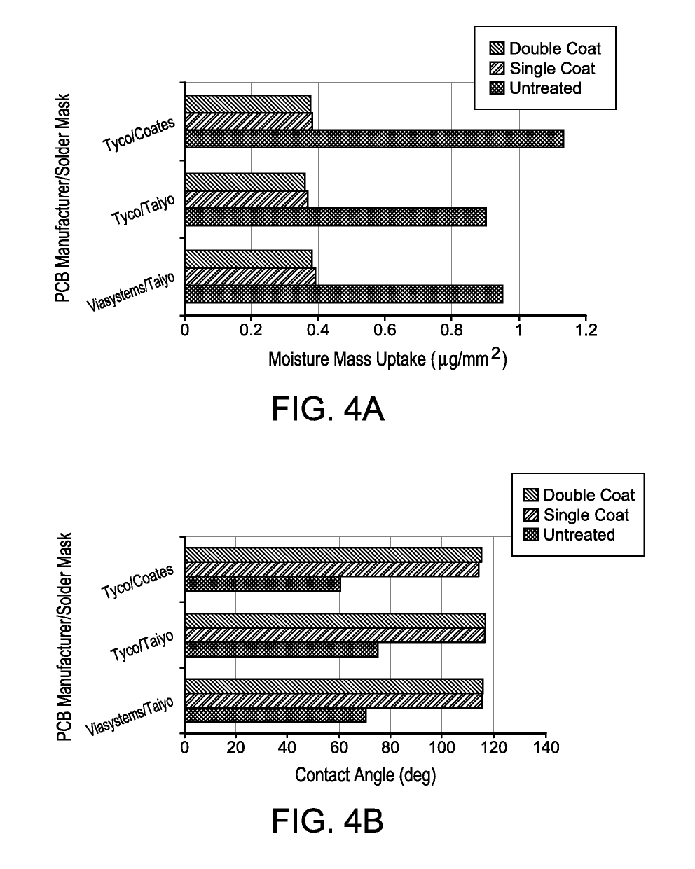

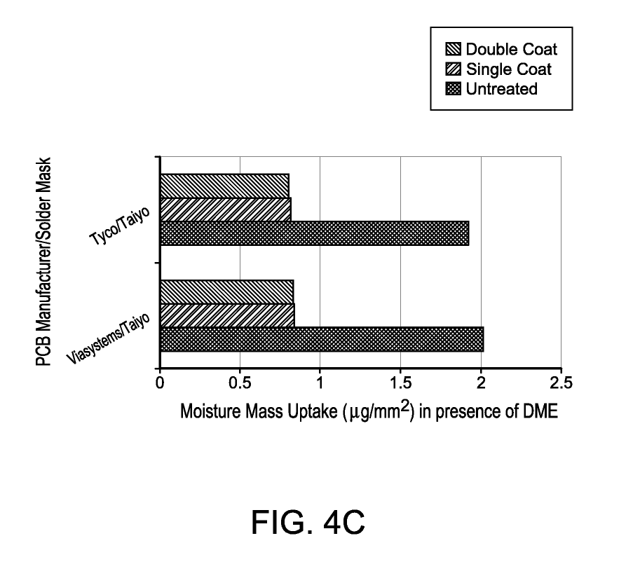

We discovered that the contact angle and amount of condensed water is independent of solder mask chemistry when the hydrophobic modifier is applied. In particular, we obtained the same preferred contact angle for a surface coated with a particular hydrophobic modifier regardless of what surface materials or solder masks was present. Similarly, the moisture mass uptake depends on the characteristics of the hydrophobic modifier, yet is independent of the surface chemistry. Even in the presence of volatile organics, such as DME, the surface chemistry has no effect on the contact angle of a hydrophobic modifier-coated surface. Furthermore, the contact angle of a hydrophobic modifier-coated surface is unchanged by the number of coated layers applied, indicating that a single layer suffices to yield the preferred contact angle. FIGS. 4A-C exemplify our findings. This is not necessarily true of other coating materials described in the prior art, as wettability of the base materials such as the solder mask do not allow as easy an application. The amount of water that condenses as well as the wettability of the surface determines the amount and size of the water droplets on the boards. Our experiments have shown that the chemistry of the solder mask, which is used to block areas of the electrical traces from exposure, has a profound effect on the wettability, reducing the amount of condensed water up to 70%. Contact angles for PCB coated with different hydrophobic modifiers for the Taiyo and Ciba solder masks are listed in Table 1.

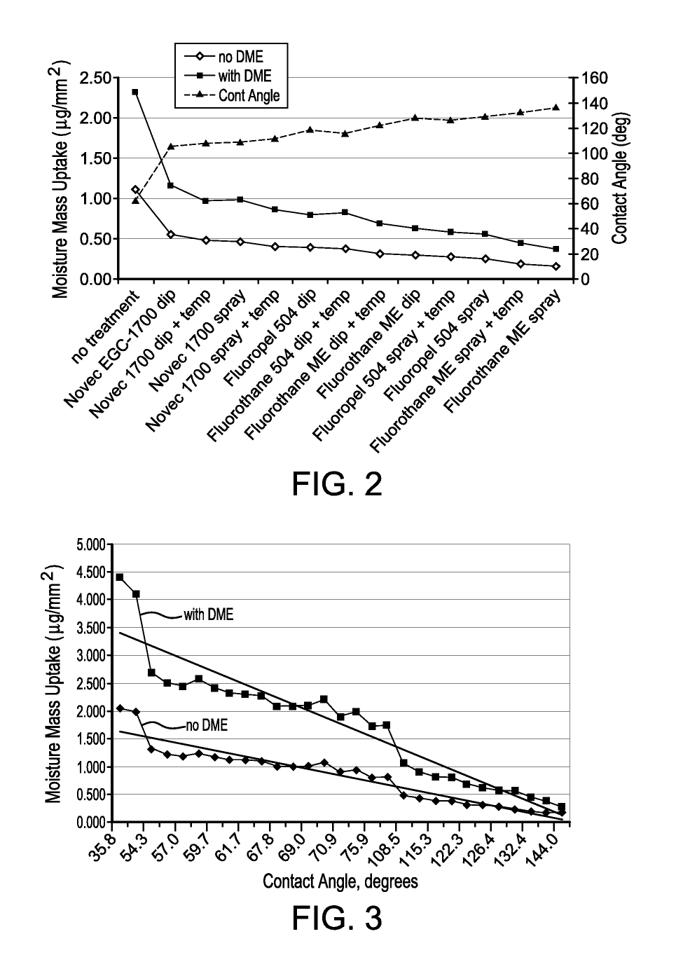

Referring to FIG. 2, an overview of exemplary embodiments of hydrophobic coatings and their respective effect on moisture condensation as measured in moisture uptake and contact angle are presented. The moisture uptake was measured after first equilibrating the samples with relative humidity of 60% at a temperature of 25° Celsius. Then the relative humidity was ramped up to 98.5% relative humidity. After allowing the samples to reequilibrate the moisture mass uptake between 60% and 98.5% relative humidity was determined in a microbalance system. A clear drop in moisture uptake is shown after applying a hydrophobic coating compared to no treatment with a hydrophobic modifier. Equivalently, a sharp increase in contact angle from 62 degree to larger than 105 degree is caused by applying a hydrophobic modifier coating. In addition, FIG. 3 demonstrates an almost linear relationship between moisture mass uptake and contact angle of surfaces possessing a wide range of hydrophobicity, regardless of the presence or absence of DME, with the moisture mass uptake approximately doubling when DME is present. Therefore, in deciding which hydrophobic modifier to apply only the contact angle needs to be considered.

Furthermore, standard materials used in conformal coatings are known to outgas many different chemical species, some of which can be extremely corrosive in a humid environment (e.g. acetic acid). In contrast, fluorochemicals used as hydrophobic coatings outgas only a small amount of material when cured, and the chemicals that do outgas are not corrosion accelerators.

TABLE 1
Contact angles of PCBs treated with various hydrophobic modifiers

| Hydrophobic Modifier Treatment | Contact Angle (degree) |
|---|---|
| no treatment | 62.3 |
| Novec EGC-1700 dip | 105.0 |
| Novec EGC-1700 dip & Ciba-mask | 106.1 |
| FluorN 562 | 106.9 |
| FluorN 562 dip & Ciba-mask | 107.3 |
| Novec 1700 dip + temp | 108.1 |
| Novec 1700 spray | 108.5 |
| FluorN 561 | 108.5 |
| Novec 1700 spray + temp | 111.1 |
| FluorN 561 dip & Ciba-mask | 114.4 |
| FluoroPel 1304A dip | 114.5 |
| FluoroPel 1304A dip & Ciba-mask | 115.0 |
| FluoroPel 504A dip + temp | 115.3 |
| FluoroPel 504A dip & Ciba-mask | 117.5 |
| FluoroPel 504A dip | 117.9 |
| Fluorothane ME dip + temp | 122.3 |
| FluoroPel 504A spray + temp | 126.4 |
| Fluorothane ME dip | 127.9 |
| FluoroPel 504A spray | 129.1 |
| Fluorothane ME dip & Ciba-mask | 132.0 |
| Fluorothane ME spray + temp | 132.4 |
| Fluorothane ME spray | 136.4 |

(temp = heated to 225° Celsius for 3 min)

The present invention is exemplified with reference to the following Example.

### Example

FluoroPel™ PFC 504A (“504A”) from Cytonix Corp., a proprietary hydrophobic modifier, has been applied to printed circuit boards manufactured by Viasystems Group Inc. or Tyco Inc. with solder masks including “Taiyo PSR4000BN(HV)” (“Taiyo”), “Ciba Probimer 52” (“Ciba”), and “Coates ImageCure XV501T-4” (“Coates”). “504A” is an inert fluoropolymer that can be applied by spray coating techniques. It dries to a thin transparent coating of approximately 2 microns thickness. Application of the phobic modifier to the printed circuit board (PCB) panels is accomplished through use of a spray-coating machine. The spray coater is equipped with a conveyer belt to feed the panels into the spray chamber on a continuous basis. Inside the chamber, a spray nozzle applies a solution containing the modifier. The modifier is dissolved in an inert fluorocarbon solvent that is highly volatile. The nozzle is able to move completely in 3 dimensions to apply modifier to both sides of the boards. It follows a pre-programmed set of commands to apply modifier only where desired, and avoid areas that need not get a coating. The solvent is sufficiently volatile so that by the time the boards exit the chamber they are completely dry and fully modified.

When “504A” is applied to the board surfaces (approximately 2400 mm² in size), it changes the surface properties to make them significantly more hydrophobic. This results in a significant reduction in moisture mass uptake due to condensation on to the boards (see FIG. 4A). The comparison of moisture uptake measurements of PCBs shows that the Tyco board coated with the “Coates” solder mask has the largest decrease of about 0.7 μg/mm². All data were measured in an environment of 98.5% relative humidity and at a temperature of 25° Celsius. The data demonstrate that the hydrophobically modified PCBs adsorb much less water than when not treated.

This is beneficial in that much less liquid water is available to promote corrosion around the board components (FIG. 1G). In addition, the coating 6 increases the surface tension, as measured by water contact angle (see FIG. 4B). This graph shows the changes in contact angle, when PCBs with either “Coates” or “Taiyo” solder masks 2 are treated with the hydrophobic modifier “504A”. When treated, the contact angle increases dramatically. This increase in contact angle corresponds to a decrease in the amount of water on the PCBs, as well as a lower amount of wetting of the water droplets on the surface. Increasing the surface tension causes water 4 that does condense on the surface to bead up rather than spread out on the board, which makes it less likely to bridge oppositely charged contacts 3, which is one contributor to corrosion (see FIG. 1G).

Exposure of the hydrophobic modifier coated PCB to solder re-flow temperature does not degrade the hydrophobic modifier performance. Such exposure increases hydrophobicity in terms of the contact angle by about 2 to 3 degree, further reducing moisture mass sorption by a few hundred μg/mm².

Each PC board manufacturer applies a different solder mask to their boards. Since the chemistry among different solder masks varies greatly, the physical surface properties of the boards also are very distinct, in particular their hydrophobic characteristic. In addition, varying the solder mask processing conditions resulted in different PCB hydrophobicities. Table 2 summarizes various combinations of these conditions leading to the PCB boards with the “Taiyo” solder mask exhibiting contact angles in the range from 54 to 79 degree. Statistical analysis revealed that a UV bump does not increase the contact angle, while changes in the temperature during the tack dry or final cure process had the most noticeable effect in increasing the hydrophobicity range of the PCBs.

TABLE 2
Condition of solder mask processing

| | Temperature (Celsius) | | Time | |
|---|---|---|---|---|
| Tack dry | 60° | 80° | 35 mins | 70 mins |
| Final Cure | 135° | 200° | 60 mins | 90 mins |

| | |
|---|---|
| UV bump | 0 J and 2.5 J for each combo of time, temp. |

Since an electrotransport drug delivery device such as the IONSYS™ system is sensitive to the amount of moisture that condenses on its printed circuit board, it is desirable to maximize the hydrophobicity of the solder mask in terms of moisture mass uptake and contact angle. Our experiments have shown that different solder masks display markedly different hydrophobicities due to changes in surface energy imparted by the mask as indicated by the “Untreated” data points in the graphs of FIGS. 4A and 4B. This complicates selection of PCBs among different manufacturers and solder masks, since hydrophobicity is an important factor. Use of a “504A” coating makes all of the solder mask chemistries we have tried equally hydrophobic (see FIG. 4A-C). In addition, the overall hydrophobicity is unchanged by the number of coated layers applied as shown by comparing a single layer hydrophobic modifier coat to a double layer coat.

Therefore, one benefit of hydrophobic modification is that the process is largely insensitive to the choice of solder mask and thickness of the hydrophobic coating applied to the PCBs. This gives a design team of a device such as IONSYS™ considerably leeway in choosing a PCB from a specific manufacturer, as the choice of solder mask becomes significantly less important.

Furthermore, we also demonstrated that the choice of solder mask has an effect on the switch failure rate in the IONSYS™ system over a device storage period of 3 months. The lower the contact angle of the solder mask, the higher the failure rate. As the contact angle decreases, the hydrophobicity of the surface decreases, which leads to a greater volume of water condensing on the surface. Applying “504A” will eliminate switch failure of the device.

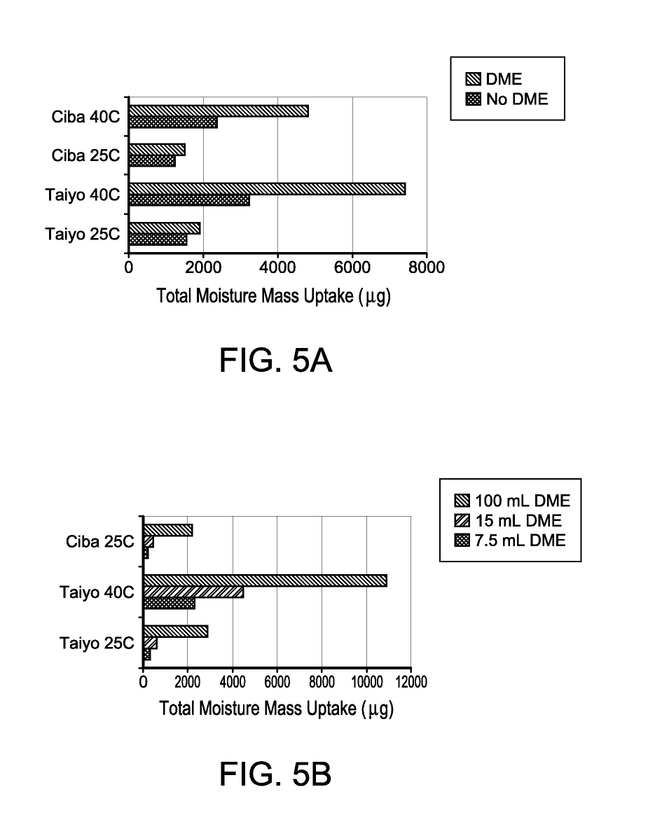

The presence of an organic volatile such as dimethoxyethane (DME) in an enclosed system clearly increases the moisture mass uptake of “untreated” PCBs as demonstrated in FIG. 5A. The larger the amount of DME injected into the enclosed headspace, the more water vapor is forced into moisture condensation (FIG. 5B). The effect that DME has on moisture uptake is more pronounced at higher temperatures (25° Celsius vs. 40° Celsius) and varies among different solder masks such as “Ciba” and “Taiyo” (FIG. 5A). Yet, applying “504A” again decreases moisture uptake drastically, even when DME is present at 95% relative humidity and 25° Celsius, yielding identical values for single and double layers of “504A” (FIG. 4C).

The above examples are illustrative in nature and are in no way intended to be limiting. While a number of exemplary aspects and embodiments have been discussed above, those of skill in the art will recognize certain modifications, permutations, additions and subcombinations thereof. It is therefore intended that the following appended claims and claims hereafter introduced are interpreted to include all such modifications, permutations, additions and subcombinations as are within their true spirit and scope.

What is claimed:

1. An electrotransport device for delivery of a beneficial agent through a body surface of a patient, comprising:
a first and second electrode assemblies, at least one of the electrode assemblies containing a beneficial agent to be delivered;
an electrical circuit electrically connectable to the first electrode assembly and to the second electrode assembly for electrically driving of the beneficial agent by electrotransport,
the electrical circuit having a hydrophobic coating and an electrical component under the hydrophobic coating, such that the electrical component is solderable and the hydrophobic coating provides for reduced water droplet condensation.

2. The device of claim 1, comprising a printed circuit board (PCB) carrying the electrical circuit, the PCB having an electrically non-conductive substrate, electrically conductive circuit paths printed on the substrate, and a solder mask covering the substrate and at least a portion of the electrically conductive circuit paths.

3. The device of claim 2, wherein the printed circuit paths are separated by spaces at least 0.22 mm wide.

4. The device of claim 2, wherein the electrical component comprises an electrical contact of the electrically conductive circuit paths.

5. The device of claim 1, wherein the hydrophobic coating is of a thickness that does not prevent water penetration to the electrical component under the hydrophobic coating, but on which a water droplet has contact angle of at least 80°.

6. The device of claim 5, wherein the contact angle is greater than about 105°.

7. The device of claim 1, wherein the hydrophobic coating contains a fluorinated polymer.

8. The device of claim 1, wherein the hydrophobic coating contains a fluorinated polymer soluble in a solvent in an amount greater than 1 wt % at room temperature.

9. The device of claim 8, wherein the solvent in which the fluorinated polymer is soluble comprises hydrofluoroether.

10. The device of claim 1, wherein the hydrophobic coating is a fused material comprising a fluorinated polymer.

11. The device of claim 1, wherein the hydrophobic coating contains a fluorinated polymer and is less than 10 microns in thickness.

12. The device of claim 11, wherein the thickness of the hydrophobic coating is between 2 to 5 microns.

13. The device of claim 1, comprising a power source that emits corrosive organic vapor.

14. The device of claim 13, wherein the corrosive organic vapor comprises dimethoxyethane.

15. The device of claim 1, wherein the device is enclosed in a moisture tight enclosure, and wherein at least one of the electrical assemblies contains water that emits water vapor within the enclosure.

16. The device of claim 15, wherein the device has a power source that emits corrosive organic vapor within the enclosure.

17. The device of claim 1, wherein the hydrophobic coating has a surface on which a water droplet has a contact angle of at least 80°, the electrical assemblies containing a hydrogel having a body surface contacting surface of at least 1.5 cm², wherein at water vapor saturation water preferentially condenses on the hydrogel surface and not on the hydrophobic coating.

18. The device of claim 7, wherein the fluorinated polymer is selected from the group consisted of polytetrafluoroethylene, fluorinated ethylene propylene polymer, polyvinylidene fluoride, fluoroacrylate, and combinations thereof.

19. A method of preserving an electrical arrangement of an electrotransport drug delivery device comprising:
coating a hydrophobic modifier on a printed circuit board to form a hydrophobic coating, the hydrophobic coating providing for reduced water droplet condensation;
making an electrical controller with the printed circuit board PCB for driving electrotransport drug flow from a drug reservoir in the device;
forming an electrotransport device having the coated PCB and at least one hydrogel reservoir;
enclosing the electrotransport device in a water-vapor-tight enclosure such that in the enclosure water vapor can transfer between the hydrogel reservoir and head space of the enclosure to equilibrate to water vapor saturation at room temperature.

20. A packaged electrotransport device for delivery of a beneficial agent through a body surface of a patient, comprising:
an electrotransport device having a first and second electrode assemblies, at least one of the electrode assemblies containing a beneficial agent in a hydrogel reservoir;
an electrical circuit electrically connectable to the first electrode assembly and to the second electrode assembly for electrically driving of the beneficial agent by electrotransport, the electrical circuit having a hydrophobic coating and an electrical component under the hydrophobic coating, such that the electrical component is solderable and the hydrophobic coating provides for reduced water droplet condensation; and
a water-vapor-tight enclosure enclosing the electrotransport device, such that water vapor can transfer between the hydrogel reservoir and head space of the enclosure to equilibrate to water vapor saturation at room temperature.
